(19) United States
(12) Patent Application Publication
(10) Pub. No.: US 2021/0386779 A1
(43) Pub. Date: Dec. 16, 2021

(54) COMPOSITIONS AND METHODS FOR DIETARY ENHANCEMENT OF IMMUNE SYSTEM FUNCTION

(71) Applicant: Leon Margolin, Beachwood, OH (US)

(72) Inventor: Leon Margolin, Beachwood, OH (US)

(21) Appl. No.: 17/245,415

(22) Filed: Apr. 30, 2021

Related U.S. Application Data
(60) Provisional application No. 63/018,355, filed on Apr. 30, 2020.
(60) Provisional application No. 63/025,119, filed on May 14, 2020.

(51) Int. Cl. A61K 33/30; A61K 31/198; A61K 31/375; A61K 31/593; A61K 31/355; A61K 31/352; A61K 31/12; A61K 31/49; A61K 9/00; A61P 31/12

(52) U.S. Cl. CPC A61K 33/30; A61K 31/198; A61K 31/375; A61K 31/593; A61P 31/12; A61K 31/352; A61K 31/12; A61K 31/49; A61K 9/0053; A61K 31/355

(57) ABSTRACT

Compositions and methods for dietary enhancement of immune system function. The compositions may include zinc(+2) and a zinc ionophore. The compositions may include zinc(+2), a first zinc ionophore and a second zinc ionophore different from the first zinc ionophore. The zinc ionophore may include quercetin. The zinc ionophore may include a quinine compound. The compositions may include one or more of l-lysine, vitamin C, vitamin D, vitamin E, curcumin and epigallocatechin gallate. The compositions may be manufactured as dietary supplements. The methods may include dosing quantities of components of the compositions. The dosing quantities have been recommended by multi-month clinical experience during the COVID-19 pandemic.

### CROSS-REFERENCE TO OTHER APPLICATIONS

This application claims the benefit of U.S. Provisional Application No. 63/018,355, filed on Apr. 30, 2020, and of U.S. Provisional Application No. 63/025,110, filed on May 14, 2020, both of which are hereby incorporated by reference herein in their entireties.

### BACKGROUND OF THE INVENTION

While advances in medical science have greatly reduced the spread and seventy of disease, certainly in developed countries, serious widespread microbial infections continue to occur, occasionally reaching lethal global pandemic levels.

Ongoing bacterial resistance even to latest-generation antibiotics is a growing concern. Similarly, viral mutation rates, particularly of RNA viruses, continue to challenge the effectiveness of vaccination programs.

RNA viruses, such as influenza-causing viruses and SARS-CoV-1 & -2-causing coronaviruses, readily mutate, with new strains sometimes having higher infectivity and/or lethality that previously encountered. As with influenza, new strains of such rapidly mutating viruses may require periodically updated vaccinations. The more infective/lethal new strains call for accurately targeted and rapidly deployed vaccines.

Coronaviruses, which induce synthesis of protected organelle-like environments within infected host cells for the viruses' RNA replication, have proven particularly difficult to counter. Thus, coronavirus-caused SARS-CoV-1, MERS-CoV and SARS-CoV-2, all of significant infectivity and lethality, have not yielded to intense efforts at their eradication. Even with concerted governmental and scientific program directed toward tempering SARS-CoV-2 since its emergence in 2019 as COVID-19, the scourge continues (as of filing of this application) to circle the globe, with devastating results.

Typically, by exposing the immune system to an attenuated virus or, as is often now feasible, only to select portions of the viral outer protein coat, vaccination provides the immune system with battle-hardening against the virus, but without the system having to endure the dangers of battling the actual, unattenuated virus. Upon subsequent exposure to the actual virus, the immune system of a healthy, successfully vaccinated person can usually respond with comprehensive anti-viral measures within a few days, generally before the virus has been able to make major inroads in its attack upon the host.

An unvaccinated immune system may typically take several weeks to develop comprehensive anti-viral measures. During the longer period, the viral attack can accelerate in scope and severity and can overwhelm the host's immune system, leading to serious debilitation and, in the extreme, death.

The critical role of nutrition in maintaining general good health and, in particular, in supporting robust immunological response to infection has long been recognized. As a relevant example, particularly timely with regard to COVID-19, we find that “[w]ith the established role of nutritional status on host immunity, the individual nutritional evaluation is probably essential to prepare someone for the SARS-CoV-II pandemic.” (See Gasmi A, Noor S, Tippairote T, et al. (2020) Individual risk management strategy and potential therapeutic options for the COVID-19 pandemic. Clinical Immunology 215:108409 DOI: 10.1016/j.clim.2020.108409)

Several micronutrients have been identified as useful in supporting immune system functioning, in particular in mediating immunological response to viral illnesses. These micronutrients include vitamins C, D3 and E, as well as l-lysine. Such micronutrients typically provide consistent but moderate immuno-enhancement. (See. e.g., Gorton H C, Jarvis K (1999) Hie effectiveness of vitamin C in preventing and relieving the symptoms of virus-induced respiratory infections. J. Manipulative Physiol. Ther. 22(8):530-533. DOI: 10.1916/s0161-4754(99)70005-9; Martineau A R, Jolliffe D A, Greenberg L. et al. (2019) Vitamin D supplementation to prevent acute respiratory infections; individual participant data meta-analysis, Health Technol. Assess. 23(2): 1-44. DOI: 10.3310/hta2302; Meitzet D O, Best T J, Zhang H, et al. (2020) Association of Vitamin D Status and Other clinical Characteristics With COVID-19 Test Results, JAMA Netw Open 3(9):e201922. DOI: 10.1001/jamanetworkopen.2020.19722; Lewis E D, Meydani S N, Wu D (2019) Regulatory role of vitamin E in the immune system and inflammation. IUBMB Life. 71(4):487-494. DOI: 10.1002/iub.1976; Sugeng M W, Adriani M, Wirjatmadi B (2015) The Effect of Zinc and Lysine Supplementation on Infection Rate and CD4 Count In Elderly. Biochem Physiol S5.002. DOI: 10.4172/2168-9652.S5-002.)

A micronutrient with recognized versatility in robustly supporting the immune system is zinc. The zinc(2+) ion, usually as incorporated into proteolytic and other enzymes, mediates numerous non-specific and specific immunological functions, with zinc dietary supplementation having been demonstrated in supporting proper development and function of immune system cells. (See, e.g., Roohani N, Harrell R, Kelishadi R, et al. (2013) Zinc and its importance for human health: An integrative review. J Res Med Sci. 18(2): 144-157. PMID: 23914218: PMCID PMC3724376; Wessels I, Rolles B, Rink L (2020) The Potential Impact of Zinc Supplementation on COVID-19 Pathogenesis. Front. Immunol 11:1722. DOI: 10.3389/fimmu.2020.01712: Skrajnowska D, Bobrowska-Korczak B. (2019) Role of Zinc in Immune System and Anti-Cancer Defense Mechanisms. Nutrients. 11(10):2273. DOI: 10.3390/null102273.)

However, there are several competing mechanisms for zinc(2+) transport between blood serum and the interior of cells. Achieving intracellular levels of zinc therapeutically effective against some infections, particularly coronavirus illnesses that can rapidly produce severe respiratory symptoms, may require an elevated zinc blood serum level that may impair other body functions. (See, e.g., Duncan A, Yacoubian C, Watson N, et al. (2015) The risk of copper deficiency in patients prescribed zinc supplements. J Clin Pathol. 68(9):723. DOI: 10.1136/jclinpath-2014-202837; Fosmire G J (1990) Zinc toxicity. Am. J. Clin. Nutr. 51(2): 225-227. DOI: 10.1093/ajcn/51.2.225.)

Zinc ionophores are substances that can enhance zinc transport into cells, generally by binding zinc(2+), the resulting complex of ionophore and bound ion enjoying a more efficient transport into the cell than does zinc(2+) otherwise. Use of zinc ionophores in conjunction with zinc supplementation may yield therapeutically effective zinc intracellular levels of zinc(2+) without requiring potentially deleterious zinc blood serum levels.

It would be desirable, therefore, to provide a composition combining a bio-assimilable form of zinc together with a zinc ionophore for use in a dietary supplement for enhancing immune system function.

Some zinc ionophores have been characterized in culture or in non-human studies, but not in vivo in humans, perhaps reflecting safety concerns associated with the ionophores. (See, e.g., teVelthuis a J W, van den Worm S H E, Sims A M C, et al. (2010) Zinc(2+) Inbihibits Coronavirus and Arterivirus RNA polymerase Activity In Vitro and Zinc ionophore Block the Replication of These Viruses in Cell Culture. PLoS Pathog 6(11):e1001176. DOI: 10.1371/journal.ppat.1001176; Ren T, Fu G H, Liu T F, et al. (2017) Toxicity and accumulation of zinc pyrithione in the liver and kidneys of Carassius auratus gibelio: association with P-glycoprotein expression. Fish Physiol Biochem. 43(1):1-9. DOI: 10.1007/s10695-016-0262-y.)

Zinc ionophores without major attendant safety concerns have been identified. Some of these may not be as effective as their more toxic counterparts in enhancing zinc transport into cells.

It would also be desirable, therefore, to provide a composition that combines a bio-assimilable form of zinc together with a first well tolerated zinc ionophore for use in a dietary supplementation regimen that also includes a second well tolerated zinc ionophore. It would further be desirable, therefore, to provide a composition that combines a bio-assimilable form of zinc together with a first well tolerated, zinc ionophore for use in a dietary supplementation regimen that also includes a second well tolerated zinc ionophore as well as several other immuno-enhancing micronutrients such as vitamins C and D3. The several other immuno-enhancing micronutrients may include vitamin E. The several other immuno-enhancing micronutrients may include l-lysine.

Typically, the more separate components comprising a dietary supplementation regimen, the greater the reluctance to undertake and reliably maintain the regimen over an extended time period. Time periods for which such regimens may be useful in providing enhancement of immune function, may include the duration of the annual flu season or of the COVID-19 pandemic.

It would yet further be desirable, therefore, to provide a composition that combines a bio-assimilable form of zinc together with a first well tolerated zinc ionophore and with a second well tolerated zinc ionophore together with other immuno-enhancing micronutrients, into a small number of supplements, preferable into a single supplement.

### BRIEF DESCRIPTION OF THE DISCLOSURE

A composition is provided combining a bio-assimilable form of zinc together with a well tolerated zinc ionophore for use in a dietary supplement for enhancing immune system function.

A composition is provided combining a bio-assimilable form of zinc together with a first well tolerated zinc ionophore for use in a dietary supplementation regimen that also includes a second well tolerated zinc ionophore.

A composition is provided combining a bio-assimilable form of zinc together with a first well tolerated zinc ionophore for use in a dietary supplementation regimen that also includes a second well tolerated zinc ionophore as well as several other immuno-enhancing micronutrients such as vitamins C and D3. The several other immune-enhancing micronutrients may include vitamin E. The several other immuno-enhancing micronutrients may include l-lysine.

A composition is provided combining a bio-assimilable form of zinc together with a first well tolerated, zinc ionophore and with a second well tolerated zinc ionophore together with other immuno-enhancing micronutrients, into a small number of supplements, preferable into a single supplement

Technical effects of the above compositions preferably include the ability to prevent and ameliorate viral illness. Other aspects and advantages of the compositions—including component amounts and regimen scheduling as strongly statistically recommended by clinical experience with the combinations of zinc, zinc ionophores and the other micronutrients over a 20-week period during the COVID-19 pandemic—will be further appreciated from the following detailed description.

### DETAILED DESCRIPTION OF THE DISCLOSURE

Early on in the COVID-19 outbreak in Ohio, our medical practice was confronted with the challenge of protecting our patients and staff from possibly deadly outcomes at the spreading infection while remaining open by government mandate. Therefore, in addition to adherence to national and state protocols aimed at minimizing infectious spread, we developed and implemented over-the counter (hereinafter, “OTC”) formulations and methods directed to prevent or, at least, ameliorate illness, in particular (retro)viral illness, with a focus on COVID-19, recommending the formulations to patients and staff.

The formulations included OTC regimens of zinc; zinc ionophores; vitamins C, D3 and E; l-lysine; and ancillary and auxiliary components. The methods included recommended dosage amounts and scheduling of administration of components as well as recommended exercises in relaxation and breathing techniques (directed to handling pandemic stress and toward respiratory robustness).

In practical implementation, we were able to track patents' and staff members' use, according to recommended amounts and scheduling, of “core formulation” omponents.” Such core formulations included: zinc together with one zinc ionophore: zinc together with one zinc ionophore, plus at least one of vitamins C, D3 and E: zinc together with two zinc ionophores, plus at least one of vitamins C, D3 and E; and several of the preceding combinations of components, also with l-lysine.

Prevalent combinations maintained by patients and staff volunteering to follow the recommendations included zinc together with at least one zinc ionophore, plus at least two of vitamins C, D3 and E.

What provided measures of the core formulations' efficacies in enhancing immune function to effect protection from vital illness, was that approximately half our patients and staff declined the recommendations (for reasons often citing anticipated or actual difficulty in maintaining the multi-component regimen over an extended time period). Thus, we had both a recommendation-compliant test group and a non-compliant control group of study subjects to follow for health outcomes of use or non use of the formulations.

The formulations and methods were based on our analysis of up-to-date biochemical and medical literature directed to our patients and staff successfully navigating the spreading pandemic's infectivity, symptomology and “new normal” everyday realities of stress, shortages, and lockdowns. Our rationale for using OTC components, beyond the practicality of their availability, went to the foundational principle of medical practice of causing no harm. We wished to minimize the likelihood of making study subjects more susceptible to infection and, in case of a subject contracting the illness, of worsening the subject's condition, by use of the formulations. Given the largely unknown medical territory we had entered in early 2020, there were, though, no guarantees of success in prevention or amelioration of the SARS-CoV-2-caused disease.

Results of use of the formulations according to the methods, as initially analyzed at a 5-week interim checkpoint, were surprisingly encouraging as to efficacy of the formulations and methods. We continued to follow both groups, monitoring for symptoms of viral illness, in particular COVID-19. The initial 5-week results, already then compelling for continued use of the formulations, were strongly and starkly statistically affirmed at 20 weeks, as presented herein below.

### Formulations and Methods

### Zinc

Beyond the general immuno-supportive characteristics of zinc presented above in BACKGROUND, zinc has several effects that recommend it for prevention and amelioration of respiratory infections and, particularly, COVID-19. These include helping maintain expression of tight-junction proteins between lung-lining muco-epithelial cells, thus blocking entry of pathogens: increasing cilia length and ciliary beat-frequency in those cells' mechanical clearance of surface “litter” such as virus particles, and repair of such function in virus-damaged lung cells. As well, zinc is important in immune response modulation, tamping down on overshooting “storms” of inflammatory immune response (thus preventing, for example, high levels of inflammatory mediators such as destructive reactive oxygen and nitrogen species) and also normalizing the ratios of diverse immune cell types. Additionally, zinc is strongly implicated in inhibiting viral binding to cell membrane ACE2 receptors used by the coronavirus to latch onto the outside of potential host cells as an essential step preparatory to entering and invading those cells; and known for its inhibiting effect on functioning of viral replication enzymes such as retroviral RNA replicase, even within intracellular protective micro-environments, thus blunting the attack by those virus particles that do gain entrance to host cells. (See, e.g., Prasad A S. (2007) Zinc: mechanisms of host defense. J Nutr. 137(5): 1345-1349. DOI: 10.1093/jn/137.5 1345: Shankar A H, Prasad A S (1998) Zinc and immune function: the biological basis of altered resistance to infection. Am. J. Clin. Nutr. 68(2 Suppl):447S-463S. DOI: 10.1093/ajcn/68.2.447S; von Bülow V. Dubben S, Engelhardt G, et al. (2007) Zinc-dependent suppression of TNF-alpha production is mediated by protein kinase A induced inhibition of Raf-1, I kappa B kinase beta, and NF-kappa B. J Immunol 179(6):4180. DOI: 10.4049/jimmunol. 179.6.4180.)

Zinc as provided in various OTC compounds was used in the study. The compounds included, but were not limited to, zinc (bis)glycinate, zinc citrate, zinc orotate and zinc picolinate. We anticipate that similar commercially available zinc products would serve as appropriate sourcing. We anticipate other available compounds and chelates of zinc to also be efficacious in the formulations to exhibit the properties and provide the benefits of zinc.

### Zinc Ionophores

In addition to our selecting OTC zinc ionophores for their enhancing transport of zinc into human cells and for their safety, another consideration for judicious selection of study zinc ionophores included the selected compounds directly conferring additional benefits, particularly immunity health benefits. To that end, the alkaloid quinine and the bioflavonoid polyphenol quercetin were selected. Other zinc ionophores that were selection candidates for their safety and additional health benefits included curcumin. Other zinc ionophores that were selection candidates for their safety and additional health benefits included epigallocatechin gallate.

### Quinine

Zinc ionophore activity has been shown in quinine and its chloroquine derivatives. (See, e.g., Ogunlana O O, Ogunlana O H, Ademowo O G (2009) Comparative in vitro assessment of the antiplasmodial activity of quinine—zinc complex and quinine sulphate. c. Res. hsEsays 4(3): 180. DOI: 10.5897/SRE9000281; Xue J. Mover A, Feng B, et al. (2014) Chloroquine is a zinc ionophore. PloS one, 9(10). e109180. DOI: 10.1371/journal.pone.0109180.)

For centuries, quinine and quinine derivatives, alkaloids from bark of Quina evergreens Cinchona (typically, Cinchona calisaya), were the primary treatment of malaria. (See. e.g., Maldonado C, Barnes C L Cornett C, et al. (2017) Phylogeny Predicts the Quantity of Amimalarial Alkaloids within the Iconic Yellow Cinchona Bark (Rubiaceae: Cinchona calisaya). Front. Plant Sci. 8:391. DOI: 10.3389/fpls.2017.00391.)

Quinine has also been shown to have an independent anti-viral activity against the COVID-19-causing coronavirus. Additionally, there is evidence of an anti-TNFα effect of quinine that may be contributory to the assumed protective anti-inflammatory effect for COVID-19 patients. For example, research on IBD patients relative to SARS-CoV-2 shows possible protective effects of anti-TNFα antibodies in Crohn's patients. (See, e.g., Li X, Zhang C, Liu L, et al. (2020) Existing bitter medicines for fighting 2019-nCoV-associated infectious diseases. FASEB J. 34(5):6008-6016. DOI: 10.1096/fj.202000502; Liu W, Qi Y, Liu L, et al. (2016) Suppression of Tumor Cell Proliferation by Quinine via the inhibition of the Tumor Necrosis Factor Receptor-associated Factor 6-AKT Interaction. Mol Med Rep 14: 2171-2179. DOI: 10.3892/mmr.2016.5492; Higgins P D R, Ng S, Danese S, et al. (2020) The Risk of SARS-CoV-2 in Immunosuppressed IBD Patients. Crohn's & Colitis: 360:2(2). DOI: 10.1093/crocol/otaa026.)

Quinine and quinine derivatives as provided in OTC extracts of Quina (Cinchona calisaya) plant bark were used in the study. Such extracts include Quina™ (NutraMedix: Jupiter, Fla. US), produced by a “proprietary [water:ethanol] extraction and enhancement process”). We anticipate that similar commercially available quina-bark-based extracts or derivatives would serve as appropriate sourcing. We anticipate other similar commercially available extracts, tinctures or other preparations of quinine-containing plants, including but not limited to Quina Roja, to also be efficacious in the formulations to exhibit the properties and provide the benefits of quinine and quinine derivatives.

### Quercetin

Zinc ionophore activity has been shown in quercetin. (See, e.g., Dabbagh-Bazarbachi H, Clergeand G, Quesada I M, et al. (2014) Zinc Ionophore Activity of Quercetin and Epigallocatechin-Gallate: From Hepa 1-6 Cells to a Liposome Model. J Agric Food Chem 13: 8085-8093. DOI: 10.1021/jf5014633.)

Quercetin has also been shown to have an independent anti-viral activity against the COVID-19-causing coronavirus. It is believed to block viruses from entering cells in the first place. An Oak Ridge National Labs/University of Tennessee study of many FDA-approved compounds presented supercomputer modeling results for inhibition by them of SARS-CoV-2 viral S-spike binding to cells. The study ranked quercetin as fifth out of twenty top performers. (See Smith M, Smith J C (2020) Repurposing Therapeutics for COVID-19: Supercomputer-Based Docking to the SARS-CoV-2 Viral Spike Protein and Viral Spike Protein-Human ACE2 Interface. ChemRxiv. Preprint. DOI: 10.26434/chemrxiv.11871402.v4.)

Studies have shown quercetin also exhibiting anti-inflammatory properties, which could help mitigate the inflammatory response of cytokine and/or bradykinin storms provoked by COVID-19. (See. e.g., Kim Y J, Park W (2016) Anti-Inflammatory Effect of Quercetin on RAW 264.7 Mouse Macrophages Induced with Polyinosinic-Polycytidylic Acid. Molecules 21(4):450. DOI: 10.3390/molecules21040450:. Cheng S C, Wu Y H, Huang W C, et al. (2019) Anti-inflammatory Property of Quercetin Through Downregulation of IGAM-1 and MMP-9 in TNF-α-activated Retinal Pigment Epithelial Cells. Cytokine: 116:48-60. DOI: 10.1016/j.cyto.2019.01.001;. Haleagrahara K Miranda-Hernandcz C, Alim A, el al. (2017) Therapeutic Effect of Quercetin in Collagen-Induced Arthritis. Biomed Pharmacother 90:38-46. DOI: 10.1016/j.biopha.2017.03.026.)

Additionally, a wide range of other anti-viral/immunity benefits of quercetin have been identified. (See. e.g., Qiu X, Kroekcr A, He S, et al. (2016) Prophylactic Efficacy of Quercetin 3-β-O-d-Glucoside Against Ebola Virus Infection. Antimicrob Agents Chemother 60(9):5182-5188. DOI: 10.1128/AAC.00307-16;. Wu W, Li R, He J, et al (2016) Quercetin as an Antiviral Agent Inhibits Influenza A Virus (IAV) Entry Viruses 8(1):6. DOI: 10.3390/v8010006;. Yi L, Li Z. Yuan K, et al. (2004) Small Molecules Blocking the Entry of Severe Acute Respiratory Syndrome Coronavirus into Host Cell. J Virol 78(20): 11334-11339, DOI: 10.1128/JVI 78.20.11334-11339.2004; Liu Y, Yu C, Ji K, et al. (2019) Quercetin Reduces TNF-α-induced Mesangial Cell Proliferation and Inhibits PTX3 Production: Involvement of NE-κB Signaling Pathway. Phytother Res 33(9):2401-2408. DOI: 10.1002/pir.6430; Liu H, Lee J I, Ahn T G (2019) Effect of quercetin on the anti-tumor activity of cisplatin in EMT6 breast tumor-bearing mice. Obstet Gynecol Sci. 62(4):242-248. DOI: 10.5468/ogs.2019.62.4.242.)

Other studied health benefits of quercetin may address some comorbidities of COVID-19 and some of the disease's sequelae. (See, e.g., Marunaka Y, Marunaka R, Sun H, et al. (2017) Actions of Quercetin, a Polyphenol, on Blood Pressure. Molecules 22: 209. DOI: 10.3390/molecules22020209; Eid H M, Haddad P S (2017) The Antidiabetic Potential of Quercetin: Underlying Mechanisms. Curr Med Chem 24:355-364. DOI: 10.2174/0929867323666160909153707; Patel R V, Mistry B M, Shindc S K, et al. (2018) Therapeutic Potential of Quercetin as a Cardiovascular Agent. Eur J Med Chem 155:889-904. DOI: 10.1016/j.ejmech 2018.06.053; Babaei F, Mirzababaei M, Nassiri-Asl M (2018) Quercetin in Food: Possible Mechanisms of Its Effect on Memory. J Food Sci: 2280-2287. DOI: 10.1111/1750-3841.14317.)

Quercetin as provided in various OTC products was used in the study. Some of the products also included bromelain. Some of the products also included vitamin C. We anticipate that similar commercially available quercetin products, with or without such added micronutrients as vitamin C or bromelain, would serve as appropriate sourcing. We anticipate various other quercetin products to also be efficacious in the formulations to exhibit the properties and provide the benefits of quercetin.

### Vitamins C, D3 and E; L-Lysine

Vitamins C, D3 and E, and l-lyisine as provided in various OTC products were used in the study. The products presented a range of high purity forms of vitamins C, D3 and E and of l-lysine. Typically, each of the products used included l-lysine or a single one of vitamins C, D3 and E. We anticipate that similar commercially available l-lysine products and vitamin products (the latter whether singly containing one of vitamins C, D3 and E, or containing a combination of two or three of the vitamins) would serve as appropriate sourcing. We anticipate other forms of vitamins C, D3 and E (whether singly containing one of vitamins C, D3 and E, or containing a combination of two or three of the vitamins) or of l-lysine to also be efficacious in the formulations to exhibit the properties and provide the benefits of those designated micronutrient(s).

### Additional Components

In various combinations outlined above, the micronutrients zinc, zinc ionophores, vitamins C, D3 and E, and l-lysine, constituted formulations that may be considered “core formulations” of the study. Study subjects were encouraged to augment the core formulations with one or more additional/ancillary components or substances. The additional/ancillary components and substances were suggested based on analysis of available biochemical, nutraceutical and medical literature.

Additional components and substances that were candidates for augmentation of the core formulations included (as parts, extracts and/or derivatives of) one or more of the following natural products: Amla (Phyllantus emblica) fruit, Garlic, Ginger, Guduchi (Tinospora cordifolia), Lemon, Lianhuaqingwenjiaonang, Licorice (Glycyrrhiza glabra), Red Reishi mushroom (Ganoderma lucidum), Olive leaf, Oregano oil. Other candidates were considered for addressing specific conditions; e.g., Bupleurum falcatum root extract for patients with pulmonary symptoms.

Ancillary components and substances that were candidates for augmentation of the core formulations included (as parts, extracts and/or derivatives of) one or more of the following natural products: Adhatoda vasica, bee propolis (Apis mellifica; e.g., extract 5:1), Holy Basil (Ocimum sanctum), Lomatium dissectum (including Lomatum “Immune support” tincture), Red Marine Algae (whole alga; e.g., extract 10:1), Self-Heal (Prunella vulgaris fruit; e.g., extract 10:1), Terminalia bellerica, Piper longum (fruit), Piper nigrum (fruit), Zingiber officinale (rhizome) (e.g., “Trikatu,” a blend of equal parts of the last three listed items).

Copper (e.g., copper orotate; copper bisglycinate) was a low-concentration ancillary component for augmentation of the core formulations, at least partly as a pre-emptive compensation for zinc-induced depletion of copper.

At the outset of the pandemic, we recommended relaxation exercises to aid in handling stress. The relaxation exercise included hot fluid therapy using select ones of the additional/ancillary components and substance, and also other herbal substances, for preparing tea infusions. The other herbal substances included Ashwagandha, Chamomile (Matricarcia recutita flower), Lemon Balm leaf (Melissa officinalis), Passion Fruit (Passiflora edulis) and Valerian root.

We recommended exercises in breathing techniques to bolster pulmonary robustness in the lace of COVID-19's respiratory symptomology. We also recommended exercises for core strength maintenance and improvement, such as isometric exercise programs that could be followed under lockdown conditions at home.

### Methods

### Categorization of Subjects

From late February/early March 2020, we recommended to patients seen in-office (and to numerous telemedicine patients) and to our staff members to follow a regimen of at least the core formulations. Of in-office patients and staff, 54 subjects started with the study's voluntary OTC regimen and, thereby, constituted the study test group (reduced to 53 subjects after the second study week by a voluntary withdrawal); while 60 declined the regimen—for reasons, when given, involving implementation's complexity and/or effort; and/or indicating barriers to implementation stemming from educational and/or socio-economic background—and constituted the study control group.

Regimen-compliant test group; 53 subjects

Non-compliant control group; 60 subjects

Subjects of the regimen-compliant test group and the non-compliant control group both met the same set of inclusion criteria, given in Table 1.

TABLE 1

| CRITERION | GROUP |
|---|---|
| Age | 30+ years (Mode: ~59) |
| Gender | ~60:40 female:male |
| Ethnicity | 30-40% African American; ~5% Latino; |
| | remainder, Caucasian |
| Temperature | afebrile |
| Oxygen saturation | 94% or higher, on room air |
| Respiration rate | 12-16 |
| Pulse | 60-100 |

Besides an even share of osteoarthritis and other chronic pain conditions, both groups had roughly the same distribution of COVID-19 comorbidities such as hypertension, coronary artery disease and type 2 diabetes meilitus. All subjects were tree of any flu-like symptoms at the start of the study.

Given the prevalence of comorbidities, the groups constitute a high risk population. As the study progressed, as presented in detail below, that adult population became increasingly multiply-exposed to COVID19-causing SARS-CoV-2 virus.

Over the study period, we monitored subjects for symptoms of flu-like illness, with an active focus on COVID-19. At the outset of the pandemic, our practice's access to COVID-19 testing was quite limited. At that point, we identified subjects as confirmed COVID-19 cases or, at least, as likely COVID-19 cases upon their earliest presentation of clinical symptoms of COVID-19; or upon their being test-confirmed as COVID cases as documented in locales and practices where testing was available. Asymptomatic/pre-symptomatic subjects—with special note taken of documented, self-reported or likely exposure to diagnosed or suspected COVID-19 cases—as well as subjects presenting symptoms of unspecified flu-like illnesses, were followed for subsequent development of COVID-19 symptoms. As testing became more available, the state/regional protocols were such that we would receive notification of individual ones among our subjects having tested positive, the testing generally conducted by a subject's primary care provider, and the notifications then sent by Ohio health agencies to all the infected patient's physicians.

### Administration Methods and Dosages

We followed subjects' oral administration, via self-administration and/or as supervised by caregiver, of components of the formulations, including via drops and powders (as mixed in liquids such as water or juice), capsules and tablets. The majority of administration compliance reporting was self-reported by subjects. Oral administration was the sole method of administration assessed in the study.

### Recommendations for Disease Prophylaxis

One dose daily of the full core formulation regimen, as per Table 2.

TABLE 2

| COMPONENT | AMOUNT | |
|---|---|---|
| zinc | 25 | mg |
| Quina ™ | 10 drops, on average (the quina-bark extract was | |
| | titrated, as tolerated by some subjects, starting | |
| | at one drop, then building up to 8-16 drops daily, | |
| | but the latter taken as two 4-8 drop half-doses | |
| | twice daily) | |
| quercetin | 400 | mg |
| vitamin C | 1000 mg (as tolerated by some subjects, | |
| | as two 500 mg half-doses twice daily) | |
| vitamin D3 | 1000 IU (25 μg) | |
| vitamin E | 400 | IU |
| l-lysine | 500 | mg |

In practical application, other amounts and proportions of these substances and components per dose may have been used by some test subjects. Such individualized dosing, while noted when possible, was not statistically assessed separately from the recommended dosing. Also not assessed was use of additional/ancillary components and methods presented above, such as 1 mg daily of copper bisglycinate taken several hours after/before the regimen dose.

Post-study, many subjects reported maintaining prevention-dosing (as taken during the study period of 20 weeks) as ongoing prophylaxis, following our suggestion to continue with the regimen over any period of acute concern of contracting COVID-19 or other viral diseases, such, as the annual influenza.

### Recommendations for Early-Stage Disease Amelioration/Alleviation

Multiples daily of the prevention-dose were administered. The multiple dose followed in the study was two; i.e., for study test group subjects presenting symptoms of flu-like illness, we followed use of two prevention doses per day, administered separately or together. On the second day of administration in those mild to moderate flu-like cases, we initiated incremental increase of the zinc over two-three days to as high as 200 mg/day, as tolerated. (Our practice's telemedicine patients, not part of the study owing to the impracticably of virus exposure and protocol compliance monitoring, who called in describing likely COVID-19 symptoms, were advised to take treatment-dosing levels of the formulations.)

At least for addressing co/secondary bacterial infection, patient and/or caregiver were recommended to consider adding, as prescribed by a treating physician, 500 mg daily azithromycin for five days, or even a course of 100 mg twice daily of doxycycline for seven days. In all cases of active clinical COVID-19 in the study, other than one course of doxycycline for one subject, no antibiotics were prescribed by primary care providers or other treating practitioners.

Treatment dosing was administered for 1-5 days or until symptoms were ameliorated/alleviated. Following symptom amelioration/alleviation, prevention-dosing was resumed.

### Subject Monitoring

Our pain management practice, providing essential medical procedures and treatments, remained open throughout the pandemic, with all CDC and Ohio State rules and guidelines for COVID-19 carefully observed. Beyond those measures, we instituted overnight and weekend in-office UV surface sterilization, using apparatus of UVB and/or UVC lamps controlled by appliance timers. (It being unclear early in the pandemic as to viability of the SARS-CoV-2 virus upon diverse surfaces, we recommended such protocols and apparatus also for home use.)

Over the study period of 20+ weeks, we monitored 113 subjects (104 in-office patients and 9 staff members) for clinical symptoms of flu-like illnesses, with subsequent differential diagnosis (and, when available, with test-confirmation) of each symptomatic subject to distinguish COVID-19 from non-COVID-19 flu-like illness. We also maintained data of reported and/or observed subject exposures to clinical and/or test-confirmed cases of COVID-19.

As we are a specially practice, primary care of the subjects as well as their other medical specialty needs were typically tended to outside our practice. This impacted our study by significantly increasing potential occurrences of subjects' exposure to carriers of COVID-19 even under lockdown restrictions (e.g., in other medical practices' waiting rooms; in transit to/from the other practices; and within and in transit to/from pharmacies). Such off-premises exposures were not likely to be consistently reported and, when reported, were not readily verifiable. The firmest exposure-counts, though likely conservatively lowballed, were based on a given subject's frequency of visits to our premises and the number of pre-quarantine COVID-positive individuals (identified and quarantined post-visit) who were present in our waiting areas during each visit of the subject and with whom the subject came in contact/proximity. Our record of to-date exposure-count for each subject represents, therefore, a likely number bracketed by low and high possibilities, with some cohorts of subjects, independent of exposure-count, having more reliably trustworthy counts than others.

A second potential impact was posed by the Ohio patient pain management contract mandating that any medicaments for infection management, such as antibiotics and antivirals, be prescribed by primary care and/or infectious disease specialists. Thus impact was negated by almost none of the symptomatic subjects being prescribed such medications (the sole exception being a single course of doxycycline for one subject), thus reducing the effect of a potentially confounding factor from our data analysis. Instead, susceptibility to COVID-19 and other viral diseases, and severity of symptoms of those diseases, could be closely linked, with statistical significance, to subjects' adherence to the study regimen of supplementation.

The OTC regimen undertaken by test group subjects included the core formulations of zinc, zinc ionophores, vitamins and l-lysine, as well as at least some of the methods. In implementation over the study period, most test group subjects reported consistent use of the recommended doses of zinc and vitamins C and E: slightly lower, additionally, consistent use of quercetin and vitamin D3, and yet somewhat fewer, but still a notable plurality, additionally, consistent use of quina bark extract and l-lysine. All study test group subjects in a high exposure sub-group, which included those staff members in frequent close contact with high-risk patients, were sure to consistently use quercetin. The high exposure sub group subjects each had six or more reported/suspected exposures to COVID-19 over the study period.

### Supplement-Specific Monitoring and Cautions

### Quinine

In light of concerns of possible complications associated with quinine, its use was monitored over the study period for cardiac complications and other side effects such as blurred vision. There was no evidence of any complications or intolerance, including in patients with significant prior cardiac history of arrhythmias or other risk factors. Monitoring tor such side effects is expected to continue over and beyond the period of quina bark extract administration.

### Quercetin

While numerous health benefits have been associated with quercetin, a caution was raised by early animal studies that pointed to quercetin toxicity in relation to benign renal tumors. We monitored for adverse effects over the study period, and received no reports nor found any clinical signs of quercetin toxicity over the study period. It appears to be appropriate to continue monitoring for oncological side effects over and beyond the period of quercetin administration.

### Combination of Quina Bark Extract and Quercetin

While there is a clear rationale for combining quina bark extract and quercetin to achieve their cumulative and possibly synergistic effect, this is, to the best of out knowledge, the first clinical study to do so. We therefore monitored all subjects over the study period, and now well beyond, for toxicity and/or side effects of the combination and have not received any reports nor seen any indicators of toxicity and/or side effects of the combination.

### Results

### Outcomes

At five weeks into the study, in early/mid-April 2020, all subjects were reported to have been exposed to the virus at least once, with some already multiply exposed. At that point, none of the 53 test group subjects had presented with COVID-19 or other flu-like illness, while six of the 60 control group subjects had so presented.

The CDC was then presenting, as a rule-of-thumb guideline, a model according to which 80% of infected individuals were estimated to remain asymptomatic and 20% to develop symptoms. But, the numbers of infected individuals to be expected in our two groups were not calculable, because the infection rate (infectivity) after an exposure to COVID-19 was not established at that point in the pandemic. Consequently, we took our clinical experience of 6/60 symptomatic control group subjects as a working measure of 10% as the likelihood of becoming symptomatic after a single exposure; i.e., a post-exposure infection rate of 10%.

By that measure, the probability of an individual remaining asymptomatic after an exposure to the virus is 1−0.10=0.90. Overall, the probability of all 53 test subjects remaining asymptomatic is given by 0.90\hat{ }53=0.0038, statistically significant at the 0.01 level (that latter indicating a 1% chance of correctness of the null hypothesis that the formulations do not account for the results).

Within the groups, cohorts of multiply exposed subjects had already developed. One such test group cohort included 8 subjects exposed at least 6 times. The probability of any individual of the cohort remaining asymptomatic after 6 exposures is given by 0.9\hat{ }6=0.53 and the probability then of all 8 cohort subjects remaining asymptomatic=0.53\hat{ }8=0.0062, statistically significant at the 0.01 level.

Given the initial statistics (and, even more simply, given the stark difference in outcomes between the test group and the control group), we were encouraged as to efficacy of the formulations in enhancing immune system function in protecting against viral illness. But, even from the start of the study, when a majority of subjects declined to follow the regimen of formulations, to us it was clearly desirable for enhancing regimen-compliance to reduce the number of capsules, tablets, pills or liquids by combining core formulation components into a much smaller number of supplements, preferably one. We began to explore avenues for such reductive combinations. At the same time, the demands of our practice and the added demands imposed by the pandemic, saw our extension of the study to 20 weeks utilizing the multi-component protocols of the first five weeks.

By the end of July 2020, the study period had extended to 20 weeks, four times the duration of the earlier interim period. Over that longer period, subjects experienced significantly greater number of episodes of exposure to clinical and/or test-confirmed cases of COVID-19 and subsequently, themselves, presented more cases of COVID-19.

As of 20 weeks, a total of two of the test group had presented symptoms of viral-like illness, versus 12 of the control group. Of the 12 control group subjects with mild to moderate to severe clinical symptoms of flu-like illness, nine were diagnosed with COVID-19 infection. Each of those nine was quarantined directly upon diagnosis, but, prior to diagnosis and quarantine, had likely increased the exposure of other study subjects to the COVID-19-causing SARS-CoV-2 corona virus. The remaining three of the 12 ill control group subjects were diagnosed with non-COVID flu-like infections.

Each of the two test group subjects with mild to moderate flu-like symptoms (neither had severe symptoms), was diagnosed to not have COVID-19 but rather an unspecified non-COVID flu-like illness. The initial test subject who had opted out of the test group by ceasing to adhere to the regimen at week two, developed confirmed COVID-19 symptoms 30 days later. (We note that various telemedicine patients who undertook the regimen of formulations and methods and who also nevertheless did contract COVID-19, reported that their physicians credited the relative mildness of the patients' cases to the regimen.)

Of note is that, of the 12 control group subjects who had developed mild to moderate to severe symptoms of flu-like illnesses (including COVID-19), were at least four who had undertaken and maintained supplementation with vitamin C (500-1000 mg daily) alone, without other core formulation components; and at least three who had undertaken and maintained supplementation with vitamin D3 (1000-2000 IU daily) alone, without other core formulation components. Also notable is that the test group subject who had stopped taking most of the core formulation supplements two weeks into the study period and developed COVID-19 symptoms a month after withdrawing from the study, had maintained vitamin C input even after opting out of the test group.

These observations demonstrate the efficacy of supplementation with zinc (25 mg daily) and zinc ionophores, and at least some of the other core formulation components, as immune system prophylaxis against COVID-19 and other flu-like illnesses: and demonstrate the relative lack of anti-flu/anti-COVID-19 prophylactic efficacy of supplementation with vitamin C or D3 in the absence of zinc and zinc ionophores.

### Statistical Analyses of 20 Week Results

### Simple Ratio

A rough measure of efficacy of the core formulations for prophylaxis against and amelioration/alleviation of symptoms of COVID-19 and other flu-like illnesses can be had by simply comparing the fraction of test group subjects who became symptomatic with any flu-like illness (2/53=0.0377) over the study period's 20+ weeks, with the corresponding fraction in the control group (12/60=0.200). The experience of the test group, by this measure, was approximately 5.3 times greater than that of the control group in being protected from any flu-like illness over the study period.

### Odds Ratio

A big-picture statistical view of the 20-week subject outcome-data is provided by an Odds Ratio Table. (For terms, definitions and calculations, see. e.g., Szumilas M (2010) Explaining Odds Ratios. J Can Acad Child Adolesc Psychiatry 19(3) 227-229. PMID: 20842279 PMCID: PMC2938757.) In Table 3, “symptomatic is taken as presenting symptoms of either non-COVID or COVID-19 illness of any flu-like illness).

TABLE 3
Odds Ratio Table
Outcomes vs Circumstances over Study Period

| | Outcome | | |
|---|---|---|---|
| | Became | Remained | Group |
| Circumstance | Symptomatic | Symptom-Free | Totals |
| Test Group Subjects | 2 | 51 | 53 |
| (Supplementation with | | | |
| core formulations) | | | |
| Control Group Subjects | 12 | 48 | 60 |
| (No zinc or zinc | | | |
| Iunophores | | | |
| Cohort Totals→ | 14 | 99 | 113 |

The study Odds Ratio (OR) calculates as OR=(2/12)/(51/48)−0.1569, with 95% Confidence Interval ends given by upper end=0.7379 and lower end=0.0334. Thus, OR=0.1569, with its 95% Confidence Interval of (0.0334, 0.7379) not spanning the value 1 (the latter datum being the sine qua non for Odds Ratio Confidence Interval statistical significance).

### Binomial Analysis

### Entire Test Group, without Incorporation of Exposure Count Information

To gain a sense of the probability of validity of the null hypothesis of obtaining the test group results without the study supplementation, the data were analyzed by binomial expansion. The probability p of any subject becoming symptomatic over the study period without supplementation may be set by the experience of the control group. In the control group, 12 of the 60 subjects experienced symptoms of unspecified flu-like illnesses (9 of 12 of which were subsequently confirmed as COVID-19), for a probability p of presentation of post-exposure flu-like symptoms of 12/60=0.2. Since all flu-like symptoms are being considered together, whether or not later confirmed as COVID symptoms, and each subject either presents flu-like symptoms or does not. the probability q of any subject remaining symptom-free is given by q=1−p=1−0.20=0.80.

In its generalized form, the binomial probability for no more than j (i.e., for j or fewer) of a group of n subjects experiencing an outcome that has probability p of occurring without introduction of the study-factor may be given as Equation 1:

$${\sum }_{i = 0}^{j} ( {nC}_{j} ) {{p}^{i} ( q )}^{( n - i )} \\ where i is an index variable going from 0 to j ; \\ {and }_{n} {C}_{i} is the combinatorial operator , bookkeeping the various possible n \text{-} subject scenarios and defined as n ! \text{/} [ i ! ( n - i ) ! ] \qquad Equation 1$$

Applying this analysis to the test group, in which, over the study period, only 2 of 53 subjects became symptomatic, the probability of two or fewer subjects becoming symptomatic as a random outcome of only the factors at play among the control group (i.e., the probability of validity of the null hypothesis of no benefit being conferred by the test group's supplementation), would be given by Equation 2:

$${\sum }_{i = 0}^{2} \{ 53 ! \text{/} [ i ! ( 53 - i ) ! ] \} {0.20}^{i} {( 0.80 )}^{( 53 - i )} = 0.000733 \qquad Equation 2$$

This analysis of study group results predicts that approximately 733 out of a million trials might produce those results without benefit of an effect of the study-factor. That result being so unlikely, at better than the 0.001 significance level, the study results provide a clear demonstration of a benefit conferred on the test group by supplementation according to the formulations and methods of this disclosure.

However, it is possible that some factor other than implementation of supplementation may be contributing to the outcomes. For instance, subjects interested and motivated to implement and maintain compliance with a multi-component supplement regimen, apparently being more health conscious than control group subjects, may indeed actually be healthier, with more robust immune systems more resistant to viral infection. Perhaps also or alternatively, health conscious subjects are more careful about minimizing their exposure to virus-carrying sources and/or about post-exposure disinfection.

An approach to compensating in the analyses for such possible confounding factors may be to assign a lower supplementation-ignoring post-exposure symptom-presentation rate to the test group than that exhibited by the control group, with the difference between the control group's actual experienced post-exposure symptom-presentation rate and the test group's new lowball assigned rate “absorbing” effects of the putative confounding factors.

Analyses were conducted for three scenarios of such low test-group post-exposure symptom-presentation rates:

A) if the test group is considered to enjoy only three-quarters the control group's demonstrated post-exposure symptom-presentation rate of 0.20, then, for the test group, q=1−p=1−(3/4)0.20=1−0.15=0.85 and the binomial expansion gives Equation 3:

$${\sum }_{i = 0}^{2} \{ 53 ! \text{/} [ i ! ( 53 - i ) ! ] \} {0.15}^{i} {( 0.85 )}^{( 53 - i )} = 0.009675 , \\ still at the 0.01 significance level . \qquad Equation 3$$

B) If the test group's post-exposure infection rate is taken to be only five-eighths the control group's 0.20, then, for the test group, q=1−p=1−0.125=0.875; and the binomial expansion gives 0.0310, no longer at the 0.01 significance level, but still at a respectable 0.05 significance level.

C) If the test group's post-exposure infection rate is taken to be only one-half the control group's 0.20, then, for the test group, q=1−p=1−0.10=0.90; and the binomial expansion gives 0.0898, no longer at the 0.05 significance level but only at the 0.1 significance level, at which the null hypothesis of ‘zero study-factor effect’ presents a 10% risk of being correct and the null hypothesis, while not strongly confirmed, cannot be rejected.

Without further information us to actual exposure-experience of subjects, one could not attach statistical significance to the teat group experience if assuming scenario C's post-exposure symptom-presentation rate of 10%. The next sub-section of Statistical Analyses, factoring in subjects' exposure-experience, demonstrates statistical significance even below 10%.

### Binomial Analysis

### With Incorporation of Exposure-Count Information

At the end of the Study period, CDC and WHO data suggested that a population infected with SARS-CoV-2 typically shows 80% asymptomatic carriers and mild symptomatic cases; 15% severe symptomatic cases; and 5% critical symptomatic cases. Taking, then, an expected approximate distribution of 80% asymptomatic/pre-symptomatic carriers and 20% symptomatic cases, the study control group's presentation of 12/60=0.20 flu-like symptom presenters would indicate that the remaining 48 symptom-free control subjects of this multiply exposed group (exposed indoors, in our facilities and in other medical practices) were asymptomatic/pre-symptomatic carriers (including, perhaps, some exposed but uninfected subjects).

Applying the same distribution to the approximately equally multiply-exposed (indoors) population of 53 subjects or the study test group, a null hypothesis of ‘zero study-factor effect.’ would yield approximately 10-11 symptomatic subjects and 42-43 asymptomatic/pre-symptomatic carriers or, conservatively, 10 symptomatic and 43 asymptomatic/pre-symptomatic carriers. That the study test group outcomes included only 2, and not 10, symptom-presenting subjects is repeat confirmation of the approximately five-fold protection seen above in the Simple Ratio subsection that seems to be conferred by the core formulations upon the test group over the control.

CDC and WHO estimates both for likelihood of post-exposure infection and for likelihood of post-infection presentation of symptoms have varied over the course of the pandemic. The range for likelihood of post-exposure infection in an indoors setting (such as our facility and that of other medical practices visited by the study subjects) ranges over approximately 20-40%; and of post-infection presentation of symptoms, over approximately 20-60%. Thus, a combined likelihood of post-exposure symptom-presentation would range over approximately 4-24%, with the high end reflected, perhaps (assuming only one exposure, on average, per subject), in our study control group's experience of 12/60=20% symptom-presentation.

The CDC and WHO estimates, while displaying wide variation, are based on sample sizes thousands times larger than our study, and also cover numerous diverse settings, averaging out localized effects. Both factors lend the estimates weighty statistical authority. Making use of a midpoint of the CDC/WHO range of post-exposure symptom-presentation. 14%, and applying it to four cohorts, a-d, of the 53·2=51 non-symptomatic subjects of our test group for whom exposure-counts were firmest, yield the following:

Probability of becoming symptomatic following a single indoor-setting exposure: 0.14 (midpoint of CDC/WHO estimates)

Probability of remaining healthy (infected but asymptomatic/pre-symptomatic or completely uninfected) subsequent to a single indoor-setting exposure: 1−0.14=0.86

Probability of remaining healthy (infected but asymptomatic/pre-symptomatic or completely uninfected) subsequent to n indoor-setting exposures: 0.86ⁿ

Probability of s cohort subjects remaining healthy (infected but asymptomatic/pre-symptomatic or completely uninfected) subsequent to a indoor-setting exposures: (0.86ⁿ)^{s}

Data and results are tabulated in Table 4

TABLE 4
Probabilities of remaining asymptomatic
or uninfected for test group cohorts

| | | | Probability Single | Probability All |
|---|---|---|---|---|
| | Subjects | Exposure- | Subject of Cohort | Cohort Subjects |
| | in Cohort | Count | Remains Healthy | Remain Healthy |
| Cohort | (s) | (n) | ((1 − p)) [symbol] | ((1 − p)ⁿ [symbol] ) |
| a | 3 | 9 | 0.86⁹ = 0.257 | (0.86⁹)³ = 0.0170 |
| b | 4 | 6 | 0.86⁶ = 0.405 | (0.86⁶)⁴ = 0.0268 |
| c | 2 | 5 | 0.86⁵ = 0.470 | (0.86⁵)² = 0.221 |
| d | 1 | 3 | 0.86³ = 0.636 | (0.86³)¹ = 0.636 |
| Totals | 10 | 23 | — | 0.86⁽²⁷⁺²⁴⁺¹⁰⁺³⁾ = |
| | | | | 0.00006643 |

[symbol]

indicates data missing or illegible when filed

The results presented in Table 4 would be strikingly more dramatic with assumption of a higher post-exposure symptom-presentation rate: and strikingly less dramatic with assumption of a lower post-exposure symptom-presentation rate.

Table 5 shows calculations using the low and high ends of the CDC/WHO estimates, as well as Table 4's mid-point.

Probability All Cohort Subjects Remain Healthy

| | | | Post-Exposure Symptom-Presentation Rate (p) | | |
|---|---|---|---|---|---|
| | | | | 0.14 | |
| | | | 0.04 | (from Table 4) | 0.24 |
| Cohort | s | n | (1 − p)ⁿ [symbol] | | |
| a | 3 | 9 | 0.332 | 0.0170 | 0.000605 |
| b | 4 | 6 | 0.375 | 0.0268 | 0.00138 |
| c | 2 | 5 | 0.665 | 0.221 | 0.0643 |
| d | 1 | 3 | 0.885 | 0.636 | 0.439 |
| Totals | 10 | 23 | 0.0733 | 0.0000643 | 0.0000000240 |

[symbol]

indicates data missing or illegible when filed

There were an additional 41 asymptomatic/pre-symptomatic or uninfected test group subjects (out of 53 total) with exposure-count estimates ranging from 1 to well beyond cohort a's 9, but with the estimates deemed less reliable than those of cohorts a-d. Factoring in another 41 asymptomatic or uninfected test group subjects, even with assuming only a single indoor-setting exposure apiece (well below the average exposure-count estimate), and even with still assuming the low end of the 4-24% post-exposure symptom-presentation rate, yields a probability for all 51 asymptomatic or uninfected test group subjects having remained symptom-free of (1−0.04)⁽²⁷⁺²⁴⁺¹⁰⁺³⁺⁴¹⁾=0.0138, comfortably within the 0.05 significance level. (Obviously, with incorporation of actual, higher exposure-count estimates for different cohorts within the 41, the significance level improves. However, a factoring-in of the control group subjects who remained asymptomatic/pre-symptomatic or uninfected, but for whom our exposure-count estimates are much less reliable, would likely offset that improvement.)

For completeness, we report that the two test group subjects who became symptomatic with unspecified non-COVID-19 flu-like illness(es) were low exposure-count subjects.

### Post-Study Considerations

### Treatment Use

Much experience with SARS-CoV-2 and its attendant COVID-19 was urgently pursued and attained by the clinical and research communities over the challenging months of 2020. With that experience, we find our OTC approach to COVID-19 prevention and amelioration/alleviation corroborated by reviews of supplementation. (See, e.g., Sahebnasagh A, Saghafi F, Avan R, et al. (2020) The prophylaxis and treatment potential of supplements for COVID-19. Eur. J. Pharmacol. 887:173530. DOI: 10.1016/j.ejphar.2020.173530.) In particular, zinc supplementation, and further in particular, zinc together with ionophore, have been studied as potentially effective prophylaxis and even as treatment measures against the pandemic disease. (See. e.g., Wessels I, Rolles B, Rink L (2020) The Potential Impact of Zinc Supplementation on COVID-19 Pathogenesis. Front Immunol. 11:1722. DOI: 10.3389/fimmu.2020.01712; Skalny A V, Rink L, Ajsuvakova O P, et al. (2020). Zinc and respiratory tract infections: Perspectives for COVID-19 (Review), International Journal of Molecular Medicine 46:17-26. DOI: 10.3892/ijmm.2020.4575; Razzaque M (2020) COVID-19 pandemic: Can maintaining optimal zinc balance enhance host resistance? Toboku J. Exp. Med. 251(3):175-181. DOI: 10.1620/tjem.251.175; Pal A, Squitti R, Picozza M, et al (2020) Zinc and COVID-19: Basis of Current Clinical Trials. Biol Trace Elem Res. 2020:1. DOI: 10.1007/s12011-020-02437-9.)

With dawning consideration of OTC regimens as part of treatment measures, we disclose an administration mode that we anticipated during the study but, fortunately, never had to implement. Particularly for medical care facilities (hospitals, nursing homes, convalescent centers), it may be desirable to apportion formulation components among various modes of administration, such as: IV or other parenteral routes for ultra-filtered solubilized and buffered zinc ion, quina bark extract, vitamin C and l-lysine; and PEG administration for quercetin and vitamins D3 and E.

The OTC formulations and methods herein disclosed have been demonstrated effective in preventing COVID-19 at one dose day; and may be considered effective in treating mild to moderate symptoms of early-stage unspecified flu-like illness (presumably also COVID) at two doses/day, with no or only minimal prescription-necessary augmentation (e.g., only azithromycin, doxycyclinc or other standard antibiotics). Thus, prophylactic use of our OTC formulations and early treatment use of the OTC formulations plus minimal antibiotic use may significantly reduce the incidence of later-stage COVID-19's moderate to severe symptoms that may require other prescribed medications that may be scarce, costly or potentially risk-bearing and/or may require in-hospital procedures. Use of our OTC formulations and methods may prevent virus spread from asymptomatic/pro-symptomatic carriers and may, thus, help address infection threats of second and subsequent waves and/or of proliferating mutant strains.

### Enhanced Compliance

Study data at 5-weeks and 20-weeks strongly suggest that zinc supplementation in the daily range of 25 mg together with zinc ionophores quina tree bark extract and quercetin, as well as vitamins C, D3 and E, and l-lysine, as administered according to study protocols, evidenced the most protective prophylactic effect against COVID-19 and other viral illnesses, while supplementation with vitamin C or vitamin D alone without zinc plus ionophore(s), may not evince any noticeable prophylactic effect. While we assumed a significant effect contributed by each of the formulations' components and substances described herein, maintaining long-term use of those components and substances was challenging to subjects.

Our practical experience at the very outset of the study and then during the study period demonstrated the difficulty for many individuals to undertake and reliably maintain a daily regimen of multiple supplements over an extended time period. Because of the importance of the multiple components of the formulations, on the one hand, and the long-term compliance difficulty subjects reported with “so many” components, on the other, we advocate adoption of a combined “one capsule” formulation. Use of a single compounded supplement, or even of a two-pill or pill-plus-liquid combination, that provides all the core formulations' components should be advantageous for prophylaxis against and treatment of COVID-19 and other flu-like illnesses.

We also anticipate formulations for individuals of low body mass. Individuals of low body mass may include juveniles. Formulations for individuals of low body mass may contain one of more of core formulation components disclosed herein, at a closing level that is a fraction of core formulations' components' dosing levels.

We also anticipate formulations for individuals already receiving, by diet and/or supplementation, one of more of the core formulations' components in a quantity approaching the core formulations' components' dosing levels disclosed herein. Formulations for individuals already receiving one of more of the core formulations' components in a quantity approaching the core formulations' components' dosing levels, may contain one of more of formulation components listed above at a dosing level that is the fraction of core formulations' components' dosing levels. Such “fractional formulations” may also be useful to individuals with dietary restriction limiting their intake of the one of more of the core formulations' components.

The fraction may be about 0.1. The fraction may be about 0.2. The fraction may be about 0.3. The fraction may be about 0.4. The fraction may be about 0.5. The fraction may be about 0.6. The fraction may be about 0.7. The fraction may be about 0.8. The fraction may be about 0.9. The fraction may be any suitable fraction. Any suitable faction may include about ¼. Any suitable faction may include about ¾.

We also anticipate formulations may contain one of more of core formulation components disclosed herein, at a dosing level that is a multiple of core formulations components' dosing levels. The multiple may be about 1.5. The multiple may be about 2. The multiple may be about 2.5. The multiple may be about 3. The multiple may be about 4. The multiple may be about 5. The multiple may be about 7. The multiple may be about 10. The multiple may be any suitable multiple. Any suitable multiple may include about 6. Any suitable faction may include about 12.

Formulations disclosed herein may be manufactured as compositions of matter.

The compositions of matter may include a zinc ionophore and a bio-assimilable form of zinc. The bio-assimilable form of zinc may include zinc(2+) ion. The compositions may include one or more of l-lysine, vitamin C, vitamin D and vitamin E.

The bio-assimilable form of zinc may be present in the compositions in a mass range of 5 mg to 100 mg. The bio-assimilable form of zinc may be present in a mass range of 10 mg to 80 mg. The bio-assimilable form of zinc may be present in a mass range of 20 mg to 30 mg.

The zinc ionophore may include quercetin. The zinc ionophore may include a quinine compound. The zinc ionophore may include curcumin. The zinc ionophore may include epigallocatechin gallate. The compositions may include two or more of the quercetin, the quinine compound, the curcumin and the epigallocatechin gallate. The compositions may include the quercetin and the quinine compound.

The quercetin may be present in the compositions in a mass range of 50 mg to 1500 mg. The quercetin may he present in a mass range of 100 mg to 1500 mg. The quercetin may be present in a mass range of 100 mg to 1000 mg. The quercetin may be present in a mass range of 300 mg to 500 mg.

The quinine compound may be derived from a quina plant. The quinine compound may be extracted from bark of the quina plant.

Formulations disclosed herein may be manufactured as dietary supplements. The dietary supplements may include at least one excipient. The excipient may be of pharmaceutical grade.

The dietary supplements may be useful for enhancing immune system function. The dietary supplements may be useful in preventing and/or ameliorating viral illness.

The dietary supplements may include a bio-assimilable form of zinc. The dietary supplements may include a first zinc ionophore. The first zinc ionophore may be a first zinc ionophore compound. The dietary supplements may include a second zinc ionophore. The second zinc ionophore may be a second zinc ionophore compound. The first zinc ionophore may be different from the second zinc ionophore. The dietary supplements may include one or more of l-lysine, vitamin C, vitamin D and vitamin E.

The first zinc ionophore may include one or more of quinine; a quinine compound; quercetin; curcumin; and epigallocatechin gallate. The second zinc ionophore may include one or more of quinine; a quinine compound; quercetin: curcumin; and epigallocatechin gallate.

All references cited in the present application are incorporated in their entirety herein by reference to the extent not inconsistent herewith.

Thus, compositions and methods for dietary enhancement of immune system function have been provided. Persons skilled in the art will appreciate that the present invention can be practiced by other than the described embodiments, which are presented for purposes of illustration rather than of limitation. The present invention is limited only by the claims that follow.

What is claimed is:

1. A composition of matter comprising: a zinc ionophore and a bio-assimilable form of zinc.

2. The composition of claim 1 wherein the bio-assimilable form of zinc includes zinc(2+) ion.

3. The composition of claim 1 wherein the bio-assimilable form of zinc is present in the composition in a mass range of 5 mg to 100 mg.

4. The composition of claim 3 wherein the bio-assimilable form of zinc is present in the composition in a mass range of 10 mg to 80 mg.

5. The composition of claim 4 wherein the bio-assimilable form of zinc is present in the composition in a mass range of 20 mg to 30 mg.

6. The composition of claim 1 wherein the zinc ionophore includes quercetin.

7. The composition of claim 6 wherein the quercetin is present in the composition in a mass range of 50 mg to 1500 mg.

8. The composition of claim 7 wherein the quercetin is present in the composition in a mass range of 100 mg to 1500 mg.

9. The composition of claim 8 wherein the quercetin is present in the composition in a mass range of 100 mg to 1000 mg.

10. The composition of claim 9 wherein the quercetin is present in the composition in a mass range of 300 mg to 500 mg.

11. The composition of claim 1 wherein the zinc ionophore includes a quinine compound.

12. The composition of claim 11 wherein the quinine compound is derived from a quina plant.

13. The composition of claim 12 wherein the quinine compound is extracted from bark of the quina plant.

14. The composition of claim 11 further comprising quercetin.

15. The composition of claim 1 further comprising one or more of l-lysine, vitamin C, vitamin D and vitamin E.

16. The composition of claim 6 further comprising one or more of l-lysine, vitamin C, vitamin D and vitamin E.

17. The composition of claim 11 further comprising one or more of l-lysine, vitamin C, vitamin D and vitamin E.

18. The composition of claim 14 further comprising one or more of l-lysine, vitamin C, vitamin D and vitamin E.

19. A dietary supplement for enhancing immune system function, the supplement comprising:
a zinc ionophore, a bio-assimilable form of zinc and a pharmaceutical grade excipient.

20. The dietary supplement of claim 19 wherein the zinc ionophore includes quercetin.

21. The dietary supplement of claim 19 wherein the zinc ionophore includes a quinine compound.

22. The dietary supplement of claim 19 wherein the zinc ionophore is a first zinc ionophore an d further comprising a second zinc ionophore, the second zinc ionophore being different from the first zinc ionophore.

23. The dietary supplement of claim 22 wherein the first zinc ionophore includes quercetin and the second zinc ionophore includes a quinine compound.

24. The dietary supplement of claim 19 further comprising one or more of l-lysine, vitamin C, vitamin D and vitamin E.

25. The dietary supplement of claim 22 further comprising one or more of l-lysine, vitamin C, vitamin D and vitamin E.

26. A dietary supplement for prevention and amelioration of viral illness, the supplement comprising:
a bio-assimilable form of zinc;
a first zinc ionophore compound;
a second zinc ionophore compound that is different from the first zinc ionophore compound; and
one or more of l-lysine, vitamin C, vitamin D and vitamin E.

27. The dietary supplement of claim 26 wherein the first zinc ionophore compound includes one or more of a quinine compound, quercetin, curcumin and epigallocatechin gallate.
